(19) United States
(12) Patent Application Publication
(10) Pub. No.: US 2017/0079602 A1
(43) Pub. Date: Mar. 23, 2017

(54) INTRAORAL SENSOR AND SENSOR INDICATOR, AND INTRAORAL RADIOGRAPHIC SYSTEM INCLUDING SAME

(71) Applicants: Rayence Co., Ltd., Gyeonggi-do (KR); VATECH EWOO Holdings Co., Ltd., Gyeonggi-do (KR)

(72) Inventors: Hyung-Keun LIM, Gyeonggi-do (KR); Jin-Pyo CHUN, Gyeonggi-do (KR)

(73) Assignees: Rayence Co., Ltd., Gyeonggi-do (KR); VATECH EWOO Holdings Co., Ltd., Gyeonggi-do (KR)

(21) Appl. No.: 15/311,179

(22) PCT Filed: May 14, 2015
(86) PCT No.: PCT/KR2015/004852

§ 371 Date: Nov. 14, 2016

(30) Foreign Application Priority Data
May 14, 2014 (KR) 10-2014-0057849

(51) Int. Cl. A61B 6/00; A61B 6/14

(52) U.S. Cl. CPC A61B 6/4429; A61B 6/145; A61B 6/56

(57) ABSTRACT

Disclosed is an intraoral radiographic system includes an intraoral sensor including a sensing surface for receiving X-rays and a coupling protrusion, and a sensor indicator including a sensor coupler configured to fasten the coupling protrusion detachably, and a support body supporting the sensor coupler. The sensor coupler is configured to be engaged with the coupling protrusion by applying an elastic force, and has a stopper preventing from sliding of the intraoral sensor.

### TECHNICAL FIELD

The present invention relates generally to an intraoral radiographic system. More particularly, the present invention relates to an intraoral radiographic system configured such that an intraoral sensor is securely mounted to a sensor indicator when taking intraoral radiographs.

### BACKGROUND ART

In general, when taking intraoral radiographs of a patient in dental clinics, and so on, the intraoral sensor is engaged with the sensor indicator, and the intraoral sensor is inserted into the patient's mouth. X-rays are then radiated onto the intraoral sensor by using an X-ray irradiation device placed outside the patient's mouth. Thereby, an intraoral structure between the X-ray irradiation device and the intraoral sensor is radiographed.

Herein, the intraoral sensor is an image sensor that senses X-rays, which are projected from an additional X-ray irradiation device and pass through an object, and converts the information contained in the X-rays into electric signals that include object image data. Further, the sensor indicator is an assistant tool for positioning the intraoral sensor at a target area in the mouth of the object, and for arranging the intraoral sensor and the X-ray irradiation device to be arrayed to face each other along a straight line.

FIG. 1 is a view illustrating a conventional intraoral radiographic system including an intraoral sensor and a conventional sensor indicator.

As shown in FIG. 1, the conventional sensor indicator 20 includes: a coupling unit 21 for coupling the intraoral sensor 10 thereto; a connecting rod 22 having a predetermined length and being vertically connected to the coupling unit 21; and a ring unit 23 to which the connecting rod 22 is vertically mounted, the ring unit facing the intraoral sensor 10 in parallel.

Herein, the conventional sensor indicator may further include an additional communication unit 24 that is connected to the intraoral sensor 10 by a wire, such as a cable, and transfers the object image data received from the intraoral sensor 10 to an additional computing device.

Meanwhile, the conventional sensor indicator 20 is configured such that the coupling unit 21, to which the intraoral sensor 10 is coupled, is engaged with the edge of the intraoral sensor 10, whereby the conventional sensor indicator blocks a detection surface receiving X-rays, which is a front surface of the intraoral sensor 10. Thus, the conventional sensor indicator is problematic in that the quality of the dental X-ray image may be degraded due to the interference from the blocked detection surface.

The conventional sensor indicator is further problematic in that a size of the sensor indicator 20 may be increased to engage the edge of the intraoral sensor 10 therewith, and may occupy a larger area than imaging area, thereby increasing inconvenience to the patient.

The conventional sensor indicator is further problematic in that the sensor indicator 20 is used by being additionally mounted to the communication unit 24, and the cable, which is connected from the intraoral sensor 10 to the communication unit 24, is exposed to the outside, thereby having a coupling structure that clutters the environment in which it is used.

### DISCLOSURE

### Technical Problem

Accordingly, the present invention has been made keeping in mind the above problems occurring in the related art, and the present invention is intended to propose an intraoral sensor and a sensor indicator, and an intraoral radiographic system including the same capable of improving the quality of a dental X-ray image through a coupling structure that does not interfere with a detection surface of the intraoral sensor.

Thus, the present invention is intended to propose an intraoral radiographic system capable of realizing a coupling structure between the intraoral sensor and the sensor indicator that does not interfere with the detection surface of the intraoral sensor.

The present invention is further intended to propose an intraoral radiographic system configured to minimize connections between elements.

Objects of the present invention are not limited to the objects mentioned above, other objects not specifically mentioned will become clear to those skilled in the art from the following description.

### Technical Solution

In order to achieve the above object, according to one aspect of the present invention, there is provided an intraoral radiographic system including: an intraoral sensor having a coupler protruded rearward on a rear surface thereof opposite to a front surface receiving X-rays; and a sensor indicator having a sensor coupler configured to be coupled with the coupler of the intraoral sensor by elastic force.

Herein, the coupler and the sensor coupler may be configured to be coupled to each other by a grip connection. The sensor coupler may include a loop-shaped grip holder, and the coupler may include a grip groove provided on a side surface thereof for being engaged with the grip holder. The grip holder may be open loop-shaped. The grip holder may be closed loop-shaped, and may include a first grip rib and a second grip rib that are configured to be detachable engaged with each other. The sensor coupler may include a cap coupled to the coupler by a snap cap type connection. The cap may be configured to be mounted to the grip holder by hinging a first end of the cap, and a second end of the cap is configured to be hooked to a corresponding part of the grip holder. The sensor indicator may include: a first end of a connecting rod connected to the sensor coupler; and a ring unit connected to a second end of the connecting rod and facing the intraoral sensor. The connecting rod may include a cable holder detachably locking a cable that is connected to the intraoral sensor. The intraoral radiographic system may further include an object image data transfer unit that is provided within the ring unit and which wirelessly transfers data received via the cable that is connected to the intraoral sensor.

In order to achieve the above object, according to another aspect of the present invention, there is provided an intraoral sensor including a coupler protruded rearward on a rear surface thereof opposite to a front surface receiving X-rays, coupler and coupled to a sensor indicator by an elastic force.

Herein, the coupler may be provided with a grip groove on at least a part of a side surface thereof, coupler and is locked to the sensor indicator by being enclosed by the sensor indicator at at least a part of the grip groove.

In order to achieve the above object, according to a further aspect of the present invention, there is provided a sensor indicator that holds a coupler protruding from a rear surface of an intraoral sensor by using an elastic force.

Herein, the sensor indicator may include a grip holder that is open loop-shaped or closed loop-shaped and which encloses at least a part of a side surface of the coupler. At least a part of the grip holder may be engaged with a grip groove formed along at least a part of the side surface of the coupler.

### Advantageous Effects

According to the present invention having the above-described characteristics, the present invention has advantageous effects as follows.

Firstly, an intraoral radiographic system according to embodiments of the present invention may be configured such that a sensor coupler of a sensor indicator is coupled to a rear surface of an intraoral sensor by a variety of coupling structures, such as grip connection. Further, the intraoral sensor may be configured to be rotatable on a plane, configured to be easily mounted and dismounted, or configured to be rotatable within a predetermined angle. Thereby, it is possible to minimize inconvenience to a patient, is possible to easily position the intraoral sensor itself, and is possible to improve the quality of the dental X-ray image without interference with a detection surface of the intraoral sensor.

Further, the cable connected to the intraoral sensor is received within the connecting rod, whereby it is possible to avoid clutter in the environment where the sensor is used, thus creating a pleasant environment for oral radiography.

Further, according to the intraoral radiographic system according to embodiments of the present invention, the ring unit is integrally provided with the object image data transfer unit that transfers the object image data from the intraoral sensor to an additional computing device by loading the object image data onto a wireless signal, whereby it is possible to minimize elements, such as cables, thus creating a pleasant environment for oral radiography and also facilitating hygienic management because connections between elements are minimized.

### DESCRIPTION OF DRAWINGS

FIG. 1 is a view illustrating a conventional intraoral radiographic system including an intraoral sensor and a conventional sensor indicator;

FIG. 2 is a schematic perspective view illustrating an intraoral radiographic system according to a first embodiment of the present invention;

FIG. 3 is a schematic perspective view illustrating an intraoral sensor of the intraoral radiographic system according to the first embodiment of the present invention;

FIG. 4 is a schematic perspective view illustrating a sensor coupler of the intraoral sensor of the intraoral radiographic system according to the first embodiment of the present invention;

FIG. 5 is a schematic view illustrating the intraoral radiographic system according to a second embodiment of the present invention;

FIG. 6 is a schematic view illustrating the intraoral radiographic system according to a third embodiment of the present invention;

FIG. 7 is a schematic view illustrating the intraoral radiographic system according to a fourth embodiment of the present invention;

FIG. 8 is a sectional view illustrating the sensor coupler according to the fourth embodiment of the present invention;

FIG. 9 is a schematic view illustrating the intraoral radiographic system according to a fifth embodiment of the present invention;

FIG. 10 is a sectional view illustrating the sensor coupler according to the fifth embodiment of the present invention;

FIG. 11 is a view illustrating the intraoral radiographic system according to a sixth embodiment of the present invention;

FIG. 12 is a sectional view illustrating the sensor coupler according to the sixth embodiment of the present invention; and

FIG. 13 is a view illustrating the intraoral radiographic system according to a seventh embodiment of the present invention.

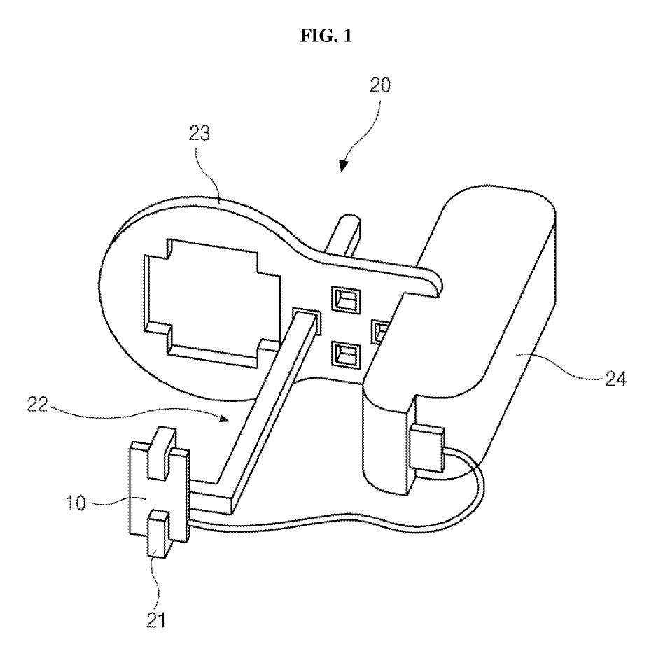

### MODE FOR INVENTION

The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms in order to describe the invention in the best way.

Firstly, in an intraoral radiographic system according to an embodiment of the present invention, a sensor indicator for intraoral radiography is configured to be coupled to an intraoral sensor by supporting a rear surface of the intraoral sensor. Further, the sensor indicator is configured to include a cable therein for being electrically connected to the intraoral sensor.

Herein, the intraoral sensor is an image sensor that senses X-rays, which are projected from an additional X-ray irradiation device and pass through an object, and converts the information contained in the X-rays into electric signals that include object image data. It is preferred that the intraoral sensor is a flexible sensor.

Reference will now be made in greater detail to an exemplary embodiment of the present invention, an example of which is illustrated in the accompanying drawings. The present invention may, however, be embodied in many different forms and should not be construed as being limited to only the embodiments set forth herein. Wherever possible, the same reference numerals will be used throughout the drawings and the description to refer to the same or like parts.

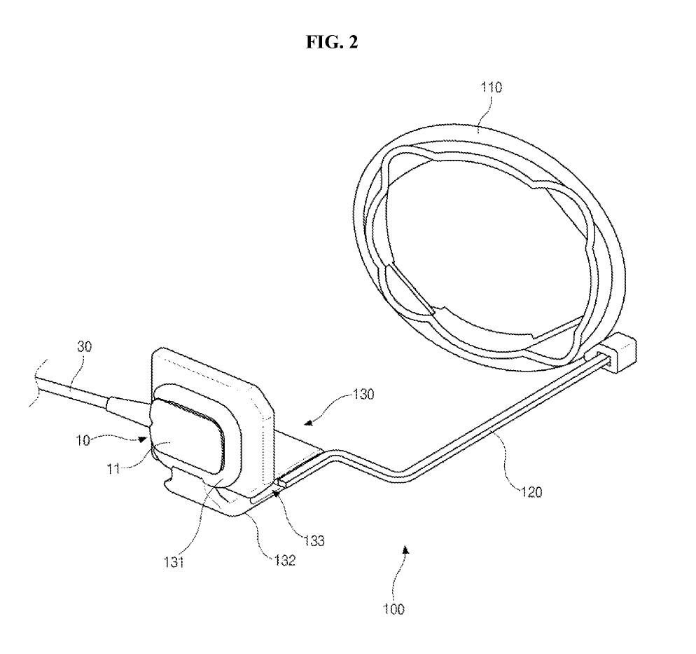

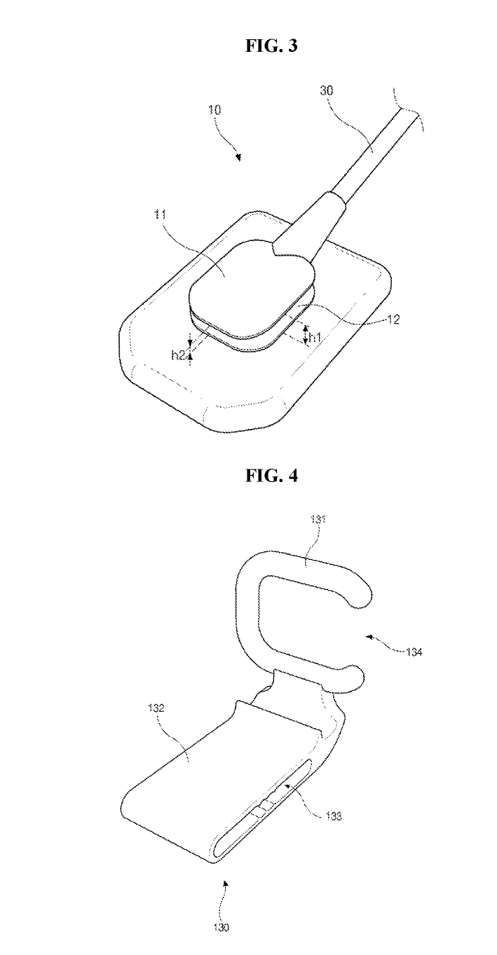

FIG. 2 is a schematic perspective view illustrating the intraoral radiographic system according to a first embodiment of the present invention; FIG. 3 is a schematic perspective view illustrating the intraoral sensor of the intraoral radiographic system according to the first embodiment of the present invention; and FIG. 4 is a schematic perspective view illustrating a sensor coupler of the intraoral sensor of the intraoral radiographic system according to the first embodiment of the present invention. Meanwhile, for the convenience of illustration, a communication unit that is connected to the cable is omitted in FIG. 2.

As shown in the accompanying drawings, the intraoral radiographic system according to the first embodiment of the present invention may include: an intraoral sensor 10; and a sensor indicator 100 to which the intraoral sensor 10 is mounted and coupled.

The intraoral sensor 10 is for producing a radiographic image by being inserted into the mouth when taking intraoral radiographs. The intraoral sensor may include an image sensor panel that converts X-rays into electric signals and an instrumental element that is connected to the image sensor panel. In particular, the intraoral sensor 10 may include a protruding grip post, namely a coupler 11 provided by protruding rearward on a rear surface thereof opposite to a sensing surface on which X-rays are incident.

The coupler 11 of the intraoral sensor 10 is configured to be detachably coupled to the sensor indicator 100, wherein the intraoral sensor 10 may be mounted and securely coupled to the sensor indicator 100 at the rear surface of the intraoral sensor via the coupler 11.

The sensor indicator 100, to which the intraoral sensor 10 is mounted, may include: a ring unit 110, a connecting rod 120, and a sensor coupler 130.

The ring unit 110 is for guiding a location of the intraoral sensor 10, and for satisfying a predetermined array standard between the sensing surface of the intraoral sensor 10 and an additional X-ray irradiation device. The ring unit may be in an annular shape with a hole having a predetermined size.

Further, the array standard may be, for example, that the detection surface of the intraoral sensor 10 and the X-ray irradiation device are arrayed to face each other along a straight line. Further, the ring unit 110 may be provided with a coupling means including a mounting groove for allowing the connecting rod 120 to be mounted thereto, and a mounting hole.

The connecting rod 120 is for connecting the ring unit 110 with the sensor coupler 130, and may be formed in a stick or in a polyhedral column shape that extends a predetermined length. The connecting rod 120 may be configured to be connected to the ring unit 110 at a first end thereof, and to be connected to the sensor coupler 130 at a first end thereof.

The connecting rod 120 may be integrally formed with the sensor coupler 130. Alternatively, the connecting rod 120 may be detachably coupled to the sensor coupler 130 by being individually provided. In the embodiment shown in FIGS, for the convenience of illustration, a case where the connecting rod 120 and the sensor coupler 130 are detachably coupled to each other is used as an example.

The connecting rod 120 may be configured such that a part thereof is inserted into the ring unit by using the coupling means provided in the ring unit 110, wherein a length of the part of the connecting rod that is inserted into the ring unit 110 is adjustable when necessary. Herein, when the length of the inserted part of the connecting rod 120 is adjusted, a separation distance between the ring unit 110 and the sensor coupler 130 may be adjustable.

The sensor coupler 130 is for supporting the rear surface of the intraoral sensor 10, and is coupled to the coupler 11 of the intraoral sensor 10. For example, through grip connection, the sensor coupler 130 is coupled to the coupler 11 of the intraoral sensor 10 by elastic force.

Reference will be made in greater detail to the coupling structure between the sensor coupler 130 and the coupler 11.

The coupler 11 that is coupled to the sensor coupler 130 may be formed into a quadrangular shape from a plan view, but not limited thereto. The coupler may be in a polygonal shape, or in other shapes. In the embodiment shown in FIGS, for the convenience of illustration, a case where the coupler 11 is in a quadrangular shape is used as an example.

The coupler 11 may be provided with a grip groove 12 on at least a part of a side surface thereof by being concavely formed so as to be coupled to the sensor coupler 130. Herein, the grip groove 12 may be formed on at least one side surface of four side surfaces of the coupler 11. For example, the grip groove 12 may be formed along three side surfaces of the coupler except one side surface thereof, through which the cable 30 is withdrawn, but not limited thereto. Meanwhile, it is preferred that the coupler 11 is provided with a grip groove 12 on opposite side surfaces thereof.

The sensor coupler 130 may include a grip holder 131 that is engaged with the grip groove 12 and is in a loop shape defined as an inner space for accommodating the coupler 11. The grip holder 131 is formed into a shape corresponding to the grip groove 12. For example, the grip holder 131 may be in an open loop shape with an opening at an edge thereof. Here, it is preferred that the opening 134 has a smaller width than a width of the coupler 11. To achieve this, each portion of the grip holder 131 meeting the opening 134 is formed into a protruding shape by being bent toward the opening 134. The bent portion of the grip holder may serve as a stop protrusion that prevents separation of the coupler 11 while the coupler 11 is coupled to the sensor coupler.

The grip holder 131 is made of an elastic material so as to be coupled to the coupler 11. For example, the grip holder may be made of a plastic material, but not limited thereto.

In order to couple the sensor coupler with the coupler 11, the coupler 11 is inserted into the grip holder 131 via the opening of the grip holder 131. Here, opposite sides of the grip holder 131 remote from each other by an elastic force thereof. When the grip holder 131 is completely inserted into the coupler, the grip holder is restored to an initial shape thereof by a restoring force, and a coupling process of the grip holder and the coupler is finished by the grip holder being engaged with the corresponding grip groove 12.

Meanwhile, the sensor coupler 130 may include a support body 132 that supports the grip holder 131 by being coupled to a lower portion of the grip holder 131. The support body 132 may be configured to be connected to the grip holder 131 at a first end thereof; and configured to extend in a direction of the front surface of the intraoral sensor 10.

Meanwhile, the support body 132 may be provided with a through hole 133 at a second end thereof, wherein the connecting rod 120 is inserted into the through hole 133 so as to be coupled to the sensor coupler 130. Here, the through hole 133 may provided with a coupling piece therein, as a coupling means coupled to an end of the connecting rod 120.

As described above, by using the grip holder 131 and the grip groove 12 that have shapes corresponding to each other, it is possible to securely couple the coupler 11 to the grip holder 131. Through grip connection, it is possible for the intraoral sensor 10 to be securely fastened to the sensor coupler 130 at the back thereof.

Thereby, it is possible to easily realize the coupling structure without interference with the front surface of the intraoral sensor 10, namely the detection surface thereof.

Meanwhile, in order to securely fasten the intraoral sensor 10 to the sensor indicator 100, a binding force between the sensor coupler 130 and the coupler 11 is required, wherein the binding force has directly to do with a geometric shape of the coupler 11.

To achieve this, through experiment, the inventors drew a numerical value for a geometric design to satisfy the required binding force of the coupler 11. To be more specific, it is preferred that an area of plan of the coupler 11 is more than 10% and less than 70% of an area of the rear surface of the intraoral sensor; that a height h1 of the coupler 11 is more than 5.0 mm; and that a height h2 of the grip groove 12 is more than 3.0 mm. Thereby, the binding force relative to the intraoral sensor 10 is satisfactory to the required level, and thus it is possible to securely couple the intraoral sensor 10 with the sensor indicator 100.

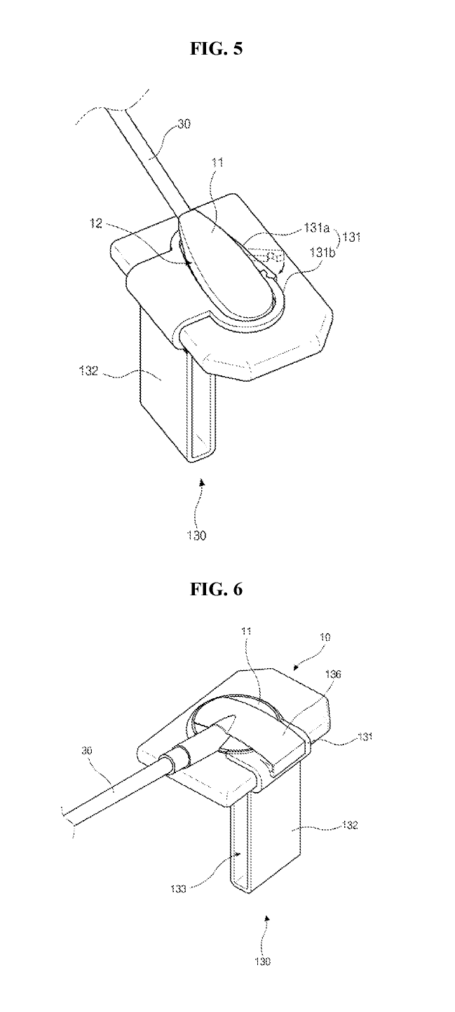

FIG. 5 is a schematic view illustrating the intraoral radiographic system according to a second embodiment of the present invention. In FIG. 5, for the convenience of illustration, the intraoral sensor 10 and the sensor coupler 130 of the sensor indicator are shown. For elements that are not shown, refer to the first and the second embodiments.

As shown in FIG. 5, in the intraoral radiographic system according to the second embodiment of the present invention, the sensor coupler 130 and the coupler 11 are coupled to each other by grip connection, in a similar manner of the first embodiment described hereinbefore.

In particular, the grip holder 131 according to the second embodiment may include a first grip rib 131a and a second grip rib 131b that are configured to be detachably engaged with each other. The first and the second grip ribs 131a and 131b may be configured to be engaged with each other at respective ends that correspond to each other by using a coupling means. For example, the first and the second grip ribs may be engaged with each other by using a hook. Thereby, when the first and the second grip ribs 131a and 131b are engaged with each other, the grip holder 131 is closed loop-shaped. In accordance with the closed loop-shaped grip holder 131, the grip groove 12 may be formed along the entire side surface of the coupler 11.

In the above case, in order to couple the coupler 11 with the grip holder, the coupler 11 is inserted through an opening provided by disengaging the first and the second grip ribs 131a and 131b from each other, and then the first and the second grip ribs 131a and 131b are engaged with each other to be closed loop-shaped. Thereby, the grip holder 131 is engaged with the grip groove 12, and the coupler 11 is coupled to the grip holder 131.

As described above, according to the second embodiment, by using the detachable grip holder 131, the grip holder 131 is engaged with the coupler 11 of the intraoral sensor 10 along the entire side surface of the coupler, thereby allowing the intraoral sensor 10 to be securely fastened thereto.

Meanwhile, in the second embodiment, a case where the coupler 11 of the intraoral sensor 10 is oval shaped is shown as an example, but not limited thereto. The coupler 11 may formed in a variety of shapes, such as an annular shape or a polygonal shape.

FIG. 6 is a schematic view illustrating the intraoral radiographic system according to a third embodiment of the present invention. In FIG. 6, for the convenience of illustration, the intraoral sensor 10 and the sensor coupler 130 of the sensor indicator are shown. For elements that are not shown, refer to the first and the second embodiments.

As shown in FIG. 6, in the intraoral radiographic system according to the third embodiment of the present invention, the coupler 11 of the intraoral sensor 10 may be coupled to the sensor coupler 130 via the grip holder 131 by grip connection, in a similar manner of the first embodiment described hereinbefore. Further, the sensor coupler 130 may include a cap 136 coupled to the coupler 11 by snap-type connection.

The cap 136 may be configured to be mounted to an corresponding part of the grip holder 131 at a first end thereof by using a hinge so as to bendable around the hinge axis. Further, the cap 136 may be provided with a hook at a second end thereof so as to be hooked to an corresponding part of the grip holder 131.

In the above case, in a state where the grip holder 131 is coupled to the coupler 11 by grip connection, the cap 136 is bendable to cover an upper surface of the coupler 11 and is hooked to the grip holder 131. Thereby, the cap 136 is coupled to the coupler 11 by a snap cap type connection.

Thus, the coupler 11 is coupled to the sensor coupler 130 by using the grip holder 131 and the cap 136, whereby it is possible to securely couple the intraoral sensor 10 with the sensor coupler.

In the first to the third embodiments described above, by using a elastic force by at least one of the grip connection and the snap cap type connection, it is possible to couple the image sensor with the sensor indicator at the back thereof.

Meanwhile, the intraoral radiographic system according to the first to the third embodiments described above may include at least some of elements in embodiments that will be described hereinbelow. For example, the intraoral radiographic system according to the first to the third embodiments may include a unit for setting the cable.

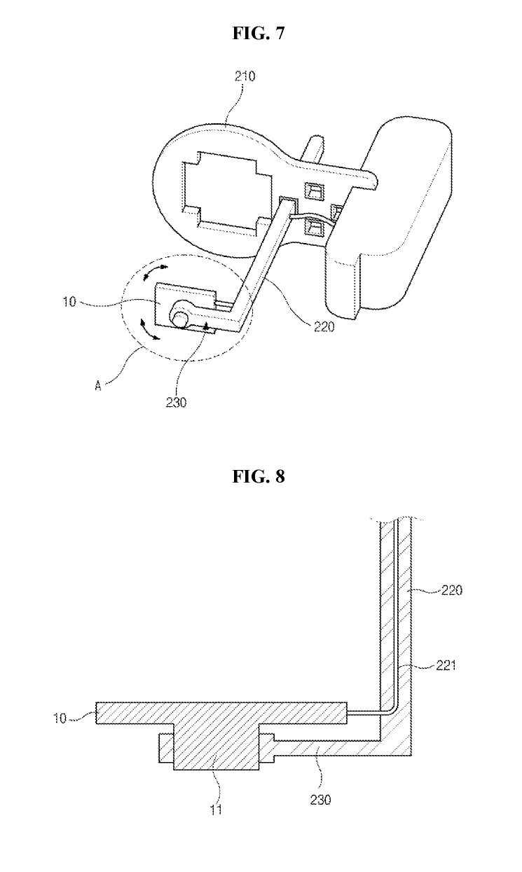

FIG. 7 is a schematic view illustrating the intraoral radiographic system according to a fourth embodiment of the present invention; and FIG. 8 is a sectional view illustrating the sensor coupler according to the fourth embodiment of the present invention.

As shown in FIGS. 7 and 8, the sensor indicator for intraoral radiography according to a fourth embodiment of the present invention is configured to be mounted to the intraoral sensor 10; and the sensor indicator includes a ring unit 210, a connecting rod 220, and a sensor coupler 230.

The ring unit 210 is for guiding a location of the intraoral sensor 10, and for satisfying a predetermined array standard between the detection surface of the intraoral sensor 10 and an additional X-ray irradiation device. The ring unit may be in an annular shape with a hole having a predetermined size.

Further, the array standard may be, for example, that the detection surface of the intraoral sensor 10 and the X-ray irradiation device are arrayed to face each other along a straight line. Further, the ring unit 210 may be provided with a coupling means including a mounting groove for allowing the connecting rod 220 to be mounted thereto, and a mounting hole.

The connecting rod 220 is for connecting the ring unit 210 with the sensor coupler 230, and may be formed in a stick or in a polyhedral column shape that extends to a predetermined length. Further, the connecting rod 220 may be integrally formed with the sensor coupler 230.

Further, the connecting rod 220 may be configured such that a part thereof is inserted into the ring unit by using the coupling means provided in the ring unit 210, wherein a length of the part of the connecting rod that is inserted into the ring unit 210 is adjustable when necessary. Herein, when the length of the inserted part of the connecting rod 220 is adjusted, a separation distance between the ring unit 210 and the sensor coupler 230 may be adjustable.

Further, the connecting rod 220 is provided with a cable holder 221 for setting or attaching the cable via which the object image data from the intraoral sensor 10 is transferred.

Herein, the cable holder 221 may be provided in a variety of manners so as to set or attach the cable to the connecting rod 220. For example, the cable holder may be configured to set and attach the cable to an outer surface of the connecting rod 220, or may be provided within the connecting rod 220.

Further, the cable holder 221 is preferably formed within the connecting rod 220 to be a hollow space for setting the cable, and is configured such that the cable for transferring data and power is provided within the connecting rod 220.

Further, the cable is electrically connected to the intraoral sensor 10 so as to supply driving power to the intraoral sensor 10 or to transfer the object image data from the intraoral sensor 10 to the outside.

The sensor coupler 230 is for supporting the rear surface of the intraoral sensor 10, and is coupled to the intraoral sensor 10. In a state where the intraoral sensor 10 is rotatably coupled to the sensor coupler, the intraoral sensor 10 is rotated in a lateral, vertical, or a diagonal direction to be located next to a tooth of the object.

Further, the sensor coupler 230 is provided with a predetermined-shaped mounting hole such that the intraoral sensor 10 is inserted thereinto. To achieve this, the rear surface of the intraoral sensor 10 may be provided with a coupler 11 that is a cylindrical protrusion.

Further, the mounting hole provided in the sensor coupler 230 may be formed into, for example, an annular shape to rotate in a state where the intraoral sensor 10 is coupled to the sensor coupler.

In other words, in the state where the intraoral sensor 10 is coupled to the sensor coupler 230, the intraoral sensor 10 may rotate. Thus, when taking intraoral radiographs, it is possible to properly rotate the intraoral sensor depending on the oral structure of the object or depending on an imaging area.

Meanwhile, not shown in the accompanying drawings, the sensor coupler 230 may include a cylindrical protrusion similar to the coupler 11; and the intraoral sensor 10 may include an annular-shaped mounting hole provided at a rear surface thereof.

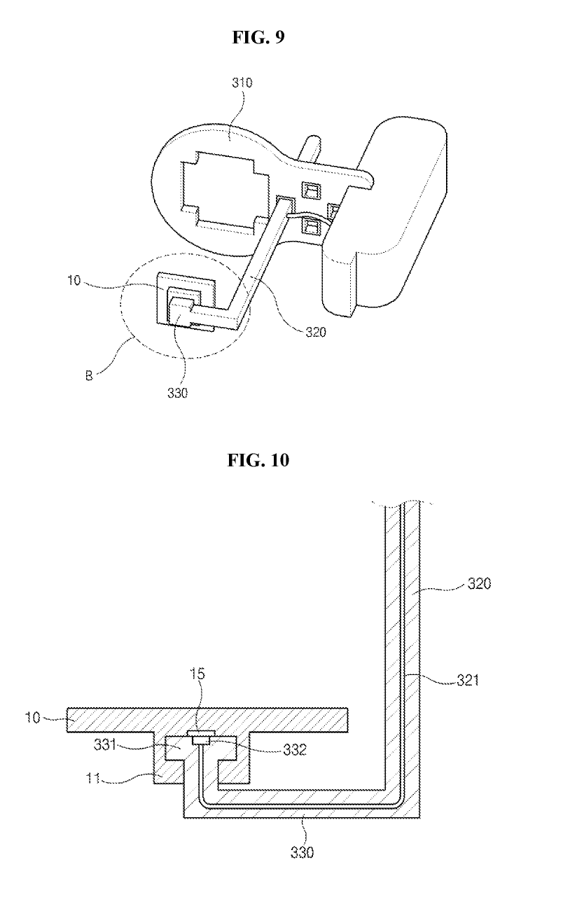

FIG. 9 is a schematic view illustrating the intraoral radiographic system according to a fifth embodiment of the present invention; and FIG. 10 is a sectional view illustrating the sensor coupler according to the fifth embodiment of the present invention.

As shown in FIGS. 9 and 10, the sensor indicator according to the fifth embodiment of the present invention is configured to be capable of mounting the intraoral sensor 10, and includes a ring unit 310, a connecting rod 320, and a sensor coupler 330.

The ring unit 310 is for arranging the intraoral sensor 10 and the X-ray irradiation device to be arrayed to face each other along a straight line. The ring unit may be in an annular shape, and may include a coupling means, to which the connecting rod 320 is mounted, provided at at least a part of the ring unit.

The connecting rod 320 is connected to the sensor coupler 330, and is mounted to the ring unit 310. The connecting rod 320 may be integrally formed with the sensor coupler, and may be formed to in a stick or in a polyhedral column shape having a predetermined length.

Further, the connecting rod 320 is provided with a cable holder 321 therein that is a hollow space such that the cable for transferring data and power is provided within the connecting rod 320. Further, the cable is electrically connected to the intraoral sensor 10 so as to transfer driving power and the object image data therethrough.

Herein, the ring unit 310 and the connecting rod 320 according to the fifth embodiment of the present invention may be configured in the same manner as the ring unit 210 and the connecting rod 220 according to the fourth embodiment of the present invention.

The sensor coupler 330 is for supporting the rear surface of the intraoral sensor 10, and is coupled to the intraoral sensor 10. The sensor coupler includes an insertion head 331 for allowing the intraoral sensor 10 to be easily and conveniently mounted to the sensor coupler.

Herein, the rear surface of the intraoral sensor 10 has a hollow space for being coupled to the insertion head 331, wherein the rear surface of the intraoral sensor may include a coupler 11 formed by protruding inward at an end thereof. Further, the insertion head 331 is coupled to the intraoral sensor 10 by being inserted into the inside of the coupler 11 of the intraoral sensor 10.

The sensor coupler 330 may further include a contact terminal 332 provided on a surface thereof and connected to the cable of the connecting rod 320. The intraoral sensor 10 may further include a connection terminal 15 for being connected to the contact terminal 332.

Preferably, the contact terminal 332 is provided in a center of an end surface of the insertion head 331 such that the connection terminal 15 of the intraoral sensor 10 and the contact terminal 332 are electrically connected to each other when the intraoral sensor 10 is coupled to the insertion head 331.

In other words, the intraoral sensor 10 is coupled and electrically connected to the sensor coupler by the simple motion of inserting the intraoral sensor 10 into the sensor coupler 330, thereby being capable of one-touch mounting and dismounting. Thus, it is possible to improve convenience, and is possible to minimize time required for intraoral photographs.

Further, the insertion head 331 of the sensor coupler 330 may be a cylinder or a column having a longitudinal section in the form of a circle or a polygon; and the coupler of the intraoral sensor 10 may be in a shape corresponding to the insertion head. In the case of having a longitudinal section in the form of a circle, the intraoral sensor 10 is rotatable relative to the insertion head 331, whereby it is possible for a user to freely move the intraoral sensor 10 in a lateral, vertical, or a diagonal direction for a purpose, and in the case of having a longitudinal section in the form of a polygon, it is possible for a user to freely move the intraoral sensor 10 in a lateral, vertical, or a diagonal direction by switching the connecting direction between the intraoral sensor 10 and the insertion head 331.

Here, the contact terminal 332 of the insertion head 331 is provided in the center of an end surface of the insertion head 331, and the connection terminal 15 of the intraoral sensor 10 is disposed at a location corresponding to the contact terminal. Thereby, even when the direction of the intraoral sensor 10 is changed, the electrical connection between the contact terminal 332 and the connection terminal 15 remains stable.

Meanwhile, on the contrary to the embodiment described above, the intraoral sensor 10 may be provided with the insertion head on the rear surface thereof, and the sensor coupler 330 may be provided with the coupler to which the insertion head is coupled.

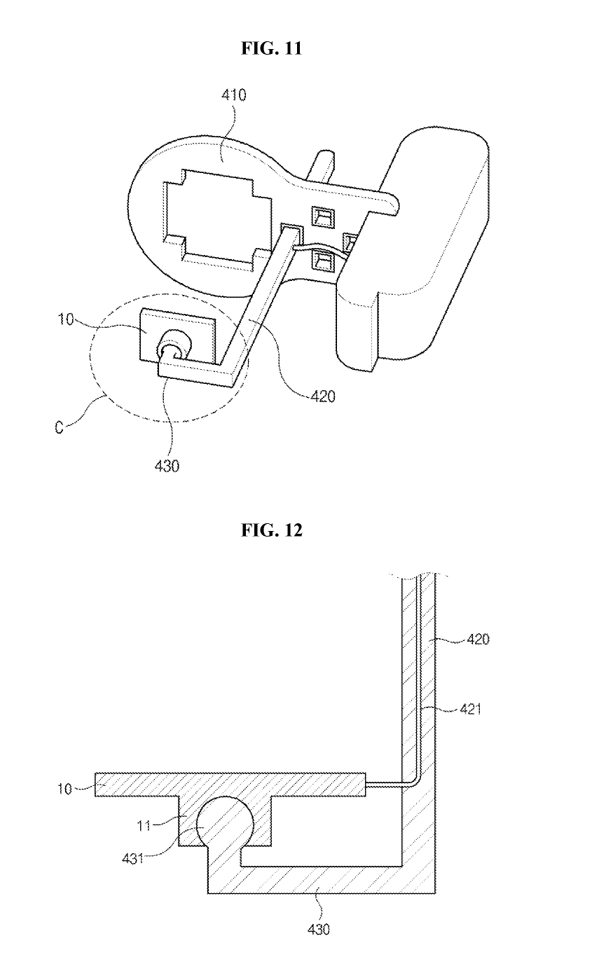

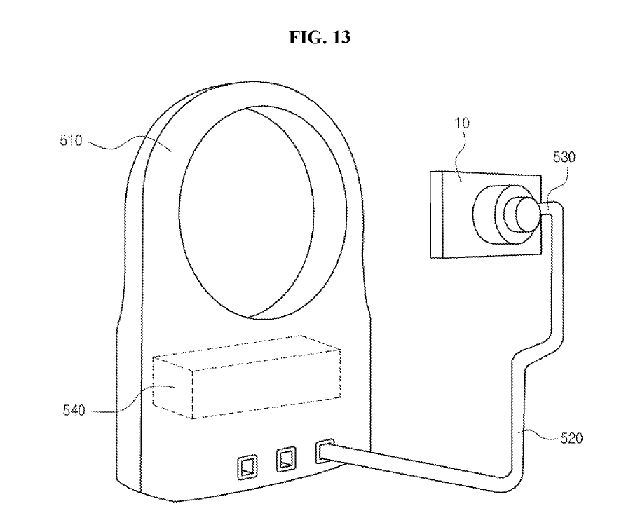

FIG. 11 is a view illustrating the intraoral radiographic system according to a sixth embodiment of the present invention; and FIG. 12 is a sectional view illustrating the sensor coupler according to the sixth embodiment of the present invention.

As shown in FIGS. 11 and 12, the sensor indicator according to the sixth embodiment of the present invention is provided for allowing the intraoral sensor 10 to be mounted thereto, and includes a ring unit 410, a connecting rod 420, and a sensor coupler 430.

The ring unit 410 is for arranging the intraoral sensor 10 and the X-ray irradiation device to be arrayed to face each other along a straight line. The ring unit is in an annular shape, and provided with a coupling means at at least a part thereof for allowing the connecting rod 420 to be mounted thereto.

The connecting rod 420 is connected to the sensor coupler 430 and mounted to the ring unit 410. The connecting rod may be integrally formed with the sensor coupler 430, and may be formed to in a stick or in a polyhedral column shape having a predetermined length.

Further, the connecting rod 420 is provided with a cable holder 421 for setting or attaching the cable that transfers data and power, wherein the cable holder 421 is formed within the connecting rod 420 to be a hollow space.

Further, the cable received in the cable holder 421 is electrically connected to the intraoral sensor 10 so as to transfer driving power and the object image data therethrough.

Herein, the ring unit 410 and the connecting rod 420 according to the sixth embodiment of the present invention may be configured in the same manner as the ring unit 210 or 310, and the connecting rod 220 or 320 according to the fourth or the fifth embodiment of the present invention.

The sensor coupler 430 is for supporting the rear surface of the intraoral sensor 10, and is coupled to the intraoral sensor 10. In a state where the sensor coupler is coupled to the intraoral sensor 10, the sensor coupler is capable of rotating within a predetermined angle.

Herein, a coupling structure between the intraoral sensor 10 and the sensor coupler 430 may be, for example, a ball joint connection that allows adjusting an angle of the intraoral sensor 10, or a hinge connection.

Further, the sensor coupler 430 includes a globular-shaped coupling protrusion 431 that is coupled to the intraoral sensor 10, and the intraoral sensor 10 includes a coupler 11 provided at a rear surface thereof by being concavely formed to correspond to the coupling protrusion 431 and to be engaged therewith.

Further, the coupling protrusion 431 is provided at an end of the sensor coupler 430, and is in a globular shape so as to allow the intraoral sensor 10 to be prevented from separation and to rotate within a predetermined angle.

Further, the intraoral sensor 10 is capable of rotating around the coupling protrusion 431 in all directions. Further, if a part of the coupler 11 of the intraoral sensor 10 is configured to be detachable, it is possible to easily mount and dismount the intraoral sensor 10.

In other words, the intraoral sensor 10 mounted to the sensor coupler 430 is capable of inclining around the coupling protrusion 431 in all directions, whereby it is possible to easily position the intraoral sensor 10 when taking intraoral radiographs.

Meanwhile, on the contrary to the embodiment described above, the intraoral sensor 10 may be provided with the coupling protrusion at the rear surface thereof, and the sensor coupler 430 may be provided with the coupler with which the coupling protrusion is engaged.

FIG. 13 is a view illustrating the intraoral radiographic system according to a seventh embodiment of the present invention.

As shown in FIG. 13, the sensor indicator according to the seventh embodiment of the present invention is provided for allowing the intraoral sensor 10 to be mounted thereto, wherein the sensor indicator includes a ring unit 510, a connecting rod 520, a sensor coupler 530, and an object image data transfer unit 540.

The ring unit 510 is for arranging the detection surface of the intraoral sensor 10 and the X-ray irradiation device to be arrayed to face each other along a straight line. The ring unit is in an annular shape, and provided with a coupling means on a side thereof for allowing the connecting rod 520 to be mounted thereto. Further, the ring unit 510 may be integrally provided with the object image data transfer unit 540 by receiving the object image data transfer unit therein.

The connecting rod 520 is connected to the sensor coupler 530 and mounted to the ring unit 510. The connecting rod may be integrally formed with the sensor coupler 530, and may be formed in a stick or in a polyhedral column shape having a predetermined length.

Further, the connecting rod 520 may further include a cable holder (not shown) that is capable of receiving the cable that transfers data and power.

The sensor coupler 530 is for supporting the rear surface of the intraoral sensor 10, wherein the sensor coupler is configured to be rotatably coupled to the intraoral sensor 10 on a plane; configured to be electrically connected and coupled to the intraoral sensor 10 by being inserted thereinto; or configured to be coupled to the intraoral sensor such that the intraoral sensor 10 is capable of rotating within a predetermined angle.

Herein, the connecting rod 520 and the sensor coupler 530 according to the seventh embodiment of the present invention may be configured in the same manner as the connecting rod 220, 320, and 420 and the sensor coupler 230, 330, and 430 according to the fourth to the sixth embodiments of the present invention.

The object image data transfer unit 540 is connected to the cable of the connecting rod 520, wherein the object image data transfer unit is electrically connected to the intraoral sensor 10 via the cable.

Further, the object image data transfer unit 540 transfers the object image data received from the intraoral sensor 10 to an additional computing device by a wire or wirelessly. It is preferred that the object image data transfer unit transfers the object image data by loading the object image data onto a wireless signal.

Further, the object image data transfer unit 540 is integrally formed with the ring unit 510. It is preferred that the object image data transfer unit is formed to be received within the ring unit 510. Further, for example, the object image data transfer unit 540 may be a wireless communication module.

In other words, the object image data communication unit 540 is configured to wirelessly communicate with the additional computing device without the cable connected to the intraoral sensor 10 exposed to the outside, and thus it is possible to minimize connections between elements without cluttering the surrounding environment of the patient.

1. An intraoral radiographic system comprising:
an intraoral sensor including a sensing surface for receiving X-rays and a coupling protrusion; and
a sensor indicator including a sensor coupler configured to fasten the coupling protrusion detachably, and a support body supporting the sensor coupler,
wherein the sensor coupler is configured to be engaged with the coupling protrusion by applying an elastic force, and has a stopper preventing from sliding of the intraoral sensor.

2. The intraoral radiographic system of claim 1, wherein the coupler is a grip holder.

3. The intraoral radiographic system of claim 1, wherein
the coupling protrusion have a groove to be engaged with the sensor coupler.

4. The intraoral radiographic system of claim 3, wherein the sensor coupler has an opening next to the stopper.

5. The intraoral radiographic system of claim 3, wherein the sensor coupler is closed loop-shaped.

6. The intraoral radiographic system of claim 3, wherein the sensor coupler includes a cap coupled to the coupling protrusion by a snap cap type connection.

7. The intraoral radiographic system of claim 6, wherein the cap is mounted to the sensor coupler by hinging a first end of the cap; and a second end of the cap is configured to be hooked to a corresponding part of the sensor coupler.

8. The intraoral radiographic system of claim 1, wherein the sensor indicator further includes a connecting rod attached to the support body, and a ring unit connected to the connecting rod and facing the intraoral sensor.

9. The intraoral radiographic system of claim 8, wherein the connecting rod includes a cable holder detachably holding a cable connected to the intraoral sensor.

10. The intraoral radiographic system of claim 8, further comprising: an object image data transfer unit provided within the ring unit and wirelessly transferring data received via the cable connected to the intraoral sensor.

11. (canceled)

12. (canceled)

13. (canceled)

14. (canceled)

15. (canceled)

16. The intraoral radiographic system of claim 8, the support body has a through hole with a coupling piece inside thereof.

17. The intraoral radiographic system of claim 1, wherein the sensor coupler is configured to be engaged with at least three side of the coupling protrusion and has an opening for a cable of the intraoral sensor.

18. The intraoral radiographic system of claim 1, wherein the sensor coupler is formed as an open-loop shape and is elastically bendable.
